SEQUENCE LISTING (2 sequences)

SEQ ID NO: 1 - DNA, 18 bp, synthetic construct
  source 1..18: mol_type = other DNA; note = Primer 27F
agagtttgacctggctag

SEQ ID NO: 2 - DNA, 18 bp, synthetic construct
  source 1..18: mol_type = other DNA; note = Primer 1495R
ctacggctccttgttcga

(19) United States
(12) Patent Application Publication
(10) Pub. No.: US 2023/0371571 A1
(43) Pub. Date: Nov. 23, 2023

(54) LACTOBACILLUS STRAIN IMAU80584, FERMENTATION MICROBIAL AGENT, AND USE THEREOF

(71) Applicant: Inner Mongolia Agricultural University, Hohhot City (CN)

(72) Inventors: Wenjun LIU, Hohhot City (CN); Jie YU, Hohhot City (CN); Jicheng WANG, Hohhot City (CN); Mei BAI, Hohhot City (CN); Guoqiang YAO, Hohhot City (CN); Zhihong SUN, Hohhot City (CN); Yongfu CHEN, Hohhot City (CN); Heping ZHANG, Hohhot City (CN)

(21) Appl. No.: 18/174,143

(22) Filed: Feb. 24, 2023

(30) Foreign Application Priority Data
May 19, 2022 (CN) 202210552581.4

(51) Int. Cl. A23L 33/135; A23C 9/123

(52) U.S. Cl. CPC A23L 33/135; A23C 9/1234

(57) ABSTRACT

The present disclosure provides a Lactobacillus strain IMAU80584, a fermentation microbial agent, and use thereof, and belongs to the technical field of microorganisms. The Lactobacillus strain IMAU80584 was deposited in the Guangdong Microbial Culture Collection Center (GDMCC), with an accession number of GDMCC 1.2566. The Lactobacillus strain IMAU80584 of the present disclosure has excellent acid-producing properties, prominent acid tolerance, and strong fermentation ability, and the Lactobacillus strain IMAU80584 exhibits a strong inhibitory effect on Escherichia coli (E. coli). As a probiotic, the Lactobacillus IMAU80584 screened by the present disclosure has promising application prospects and huge economic values in the fields of pathogenic bacteria control and fermented foods.

### CROSS-REFERENCE TO RELATED APPLICATION

This patent application claims the benefit and priority of Chinese Patent Application No. 202210552581.4, filed with the China National Intellectual Property Administration on May 19, 2022, the disclosure of which is incorporated by reference herein in its entirety as part of the present application

### REFERENCE TO SEQUENCE LISTING

A computer readable XML file entitled “GWP20220801400_seqlist. xml”, that was created on Dec. 9, 2022, with a file size of about 4,252 bytes, contains the sequence listing for this application, has been filed with this application, and is hereby incorporated by reference in its entirety.

### TECHNICAL FIELD

The present disclosure belongs to the technical field of microorganisms, and in particular relates to a Lactobacillus strain IMAU80584, a fermentation microbial agent, and use thereof.

### BACKGROUND

Probiotics, also known as microecological modulators and live bacteria preparations, refer to live microbial agents that can promote the ecological balance of intestinal floras in a host and have a beneficial effect for the host. There are a large number of microorganisms in the human gut, including beneficial bacteria, harmful bacteria, and insignificant bacteria. Under normal conditions, intestinal floras are in a balanced state, depend on and restrict each other, and coexist with the human body in a mutually beneficial manner. Intestinal flora imbalance can lead to a range of biological dysfunctions, including diarrhea and infection.

Escherichia coli (E. coli), a Gram-negative brevibacterium, is a normal flora in the human and animal intestinal tracts under normal conditions, which is motile and has peritrichous flagella and no spore. However, a small number of special types of E. coli have quite strong virulence, such as enterotoxigenic E. coli (ETEC), enterohemorrhagic E. coli (EHEC), and enteropathogenic E. coli (EPEC); and once being infected with these types, organisms, especially infants and young animals (poultry), will be subjected to severe diarrhea and sepsis, and even death. For example, after being infected with EHEC, the human body will undergo severe

spasmodic abdominal pain and recurrent hemorrhagic diarrhea, accompanied by fever, vomiting, and other manifestations; and some seriously-infected subjects undergo kidney damage and are prone to acute renal failure (ARF) and even death.

The acid-producing properties of Lactobacillus are important properties for screening fermentation agents for fermented dairy products. Fermentation agents for sour cream, fermented single cream, fermented dairy products, and similar products need to have prominent acid-producing properties. The current acidic products are most prepared through the combination of Streptococcus thermophilus (S. thermophilus) and Lactobacillus bulgaricus (L. bulgaricus), but the combination of the two is intolerant to gastric juice, intestinal juice, and bile, is difficult to colonize in an intestinal tract with an extremely-low survival rate, and can hardly play a probiotic role. Therefore, it is of great research significance and value to screen acid-producing Lactobacillus with an efficient bactericidal effect.

### SUMMARY

In view of this, the present disclosure is intended to provide a Lactobacillus strain IMAU80584, which has excellent acid-producing properties and can effectively inhibit pathogenic bacteria.

To achieve the above objective, the present disclosure provides the following technical solutions.

The present disclosure provides a Lactobacillus strain IMAU80584 deposited in the Guangdong Microbial Culture Collection Center (GDMCC), with an accession number of GDMCC 1.2566.

Preferably, growth characteristics of the Lactobacillus strain IMAU80584 are as follows: growth temperature: 10° C. to 45° C., pH: 3.5 to 9.5, and NaCl concentration: 0% to 6%.

Preferably, a fermentation substrate for the Lactobacillus strain IMAU80584 is one or more selected from the group consisting of L-arabinose, D-ribose, D-xylose, esculin-iron citrate, D-maltose, D-sucrose, and D-melezitose.

Preferably, an enzyme expressed in the Lactobacillus strain IMAU80584 is selected from the group consisting of leucine arylamidase, valine arylamidase, naphthol-AS-BI-phosphohydrolase, β-galactosidase, β-glucuronidase, and α-glucosidase.

The present disclosure also provides a fermentation microbial agent including the Lactobacillus strain IMAU80584 described above.

Preferably, an effective viable concentration of the Lactobacillus strain IMAU80584 is 1×10⁶ CFU/mL to 1×10⁸ CFU/mL.

The present disclosure also provides use of the Lactobacillus strain IMAU80584 described above or the fermentation microbial agent described above in the preparation of a fermented milk.

Preferably, the fermented milk includes a fermented soybean milk or cow milk.

The present disclosure also provides use of the Lactobacillus strain IMAU80584 described above in the preparation of a product for preventing and treating pathogenic bacteria.

Preferably, the pathogenic bacteria include E. coli.

Compared with the prior art, the present disclosure has the following beneficial effects:

The Lactobacillus strain IMAU80584 of the present disclosure is isolated from the traditional naturally-fermented dairy product (sour yak milk) in Saichi Village, Sangke Township, Xiahe County, Gansu Province, China. Test results show that the Lactobacillus strain IMAU80584 of the present disclosure has excellent acid-producing properties, prominent acid tolerance, and strong fermentation ability; the Lactobacillus strain IMAU80584 exhibits a strong inhibitory effect on E. coli; and as a probiotic, the Lactobacillus IMAU80584 screened by the present disclosure has promising application prospects and huge economic values in the fields of pathogenic bacteria control and fermented foods.

Deposit Information:

The Lactobacillus IMAU80584 (Lentilactobacillus rapi subsp. dabitei) provided by the present disclosure was deposited in the Guangdong Microbial Culture Collection Center (GDMCC) on Apr. 10, 2021, with an accession number of GDMCC 1.2566, and the Guangdong Microbial Culture Collection Center (GDMCC) is located at No. 100 Xianliezhong Road, Guangzhou, China, postcode: 510070.

### BRIEF DESCRIPTION OF THE DRAWINGS

FIG. 1 shows the colonial morphology of the Lactobacillus IMAU80584 (Lentilactobacillus rapi subsp. dabitei) in Example 1;

FIG. 2 shows a microscopic image of the Lactobacillus IMAU80584 (Lentilactobacillus rapi subsp. dabitei) in Example 1 after Gram staining;

FIG. 3 shows a transmission electron microscopy (TEM) image of a bacterial cell of the Lactobacillus IMAU80584 (Lentilactobacillus rapi subsp. dabitei) in Example 1;

FIG. 4 shows the detection results of metabolism of API 50CHL carbohydrates in the Lactobacillus IMAU80584 (Lentilactobacillus rapi subsp. dabitei) in Example 1;

FIG. 5 shows the detection results of API-ZYM enzyme activity in the Lactobacillus IMAU80584 (Lentilactobacillus rapi subsp. dabitei) in Example 1;

FIG. 6A-B show phylogenetic trees of the Lactobacillus IMAU80584 (Lentilactobacillus rapi subsp. dabitei) in Example 1 and all type strains of sibling species based on sequences of two housekeeping genes (rpoA and pheS);

FIG. 7 shows a phylogenetic tree of the Lactobacillus IMAU80584 (Lentilactobacillus rapi subsp. dabitei) in Example 1 and all type strains of sibling species based on genome-wide core genes;

FIG. 8 shows the Oxford cup antibacterial effect of a mixed bacterial solution of Lactobacillus IMAU80584 (Lentilactobacillus rapi subsp. dabitei) on E. coli in Example 4;

FIG. 9 shows the Oxford cup antibacterial effect of a bacterium-free supernatant of Lactobacillus IMAU80584 (Lentilactobacillus rapi subsp. dabitei) on E. coli in Example 4.

### DETAILED DESCRIPTION OF THE EMBODIMENTS

The present disclosure provides a Lactobacillus strain IMAU80584 deposited in the Guangdong Microbial Culture Collection Center (GDMCC), with an accession number of GDMCC 1.2566.

In the present disclosure, the Lactobacillus IMAU80584 (classification designation: Lentilactobacillus rapi subsp. dabitei) was deposited in the Guangdong Microbial Culture Collection Center (GDMCC) on Apr. 10, 2021, with an accession number of GDMCC 1.2566, and the Guangdong Microbial Culture Collection Center (GDMCC) is located at No. 100 Xianliezhong Road, Guangzhou, China, postcode: 510070.

The Lactobacillus IMAU80584 provided by the present disclosure is derived from the traditional naturally-fermented dairy product (sour yak milk) in Saichi Village, Sangke Township, Xiahe County, Gansu Province, China, and the Lactobacillus IMAU80584 has the following microbiological characteristics:

(1) Colony characteristics: A single colony has a diameter of 1 mm to 2 mm, is beige and opaque, and has a smooth and flat surface with a slightly-rough edge.

(2) Morphological characteristics: Bacterial cells are in a short-rod shape with a length of 1.67 μm to 1.91 μm and a width of 0.67 μm to 0.83 μm, are immotile in a single-rod arrangement, have no flagella, and are positive for Gram staining.

(3) Growth characteristics: The Lactobacillus IMAU80584 has a minimum growth temperature of 10° C., a maximum growth temperature of 45° C., and an optimal growth temperature of 30° C. to 37° C.; the Lactobacillus IMAU80584 has a maximum growth pH of 9.5 and a minimum growth pH of 3.5; and the Lactobacillus IMAU80584 can grow in a NaCl solution with a mass-to-volume fraction of 0% to 6%, but undergoes the optimal growth in a NaCl solution with a mass-to-volume fraction of 0% to 5%.

In the present disclosure, a fermentation substrate for the Lactobacillus strain IMAU80584 is preferably one or more selected from the group consisting of L-arabinose, D-ribose, D-xylose, esculin-iron citrate, D-maltose, D-sucrose, and D-melezitose.

In the present disclosure, an enzyme expressed in the Lactobacillus strain IMAU80584 is preferably selected from the group consisting of leucine arylamidase, valine arylamidase, naphthol-AS-BI-phosphohydrolase, β-galactosidase, β-glucuronidase, and α-glucosidase.

The present disclosure also provides a fermentation microbial agent including the Lactobacillus strain IMAU80584 described above.

In the present disclosure, an effective viable concentration of the Lactobacillus strain IMAU80584 is preferably 1×10⁶ CFU/mL to 1×10⁸ CFU/mL and more preferably 1×10⁷ CFU/mL. The fermentation microbial agent of the present disclosure further includes an adjuvant; a mass percentage of the adjuvant in the fermentation microbial agent is 4% to 8%; and the adjuvant includes one or more selected from the group consisting of Europe corn starch, sucrose, lactose, sodium glutamate, skimmed milk powder, water, glycerin, maltodextrin, soybean protein isolate (SPI), and sodium ascorbate.

The present disclosure also provides use of the Lactobacillus strain IMAU80584 described above or the fermentation microbial agent described above in the preparation of a fermented milk.

In the present disclosure, the fermented milk includes preferably a fermented soybean milk or cow milk and more preferably a fermented soybean milk. The Lactobacillus strain IMAU80584 of the present disclosure exhibits high acid-producing ability and acid tolerance, and has promising application prospects in the dairy industry.

The present disclosure also provides use of the Lactobacillus strain IMAU80584 described above in the preparation of a product for preventing and treating pathogenic bacteria.

In the present disclosure, the pathogenic bacteria preferably include E. coli. The Lactobacillus strain IMAU80584 of the present disclosure has a strong inhibitory effect on E. coli, and can improve the microecological environment in an organism and enhance the immunity.

The technical solutions provided by the present disclosure will be described in detail below with reference to examples, but the examples should not be construed as limiting the protection scope of the present disclosure.

### Example 1

Isolation and Identification of Lactobacillus IMAU80584 (Lentilactobacillus Rapi Subsp. Dabitei)

I. Isolation of Lactobacillus IMAU80584

The Lactobacillus IMAU80584 (Lentilactobacillus rapi subsp. dabitei) was isolated from the traditional naturally-fermented dairy product (sour yak milk) in Saichi Village, Sangke Township, Xiahe County, Gansu Province, China, and a specific isolation process was as follows:

A sour yak milk sample was 10-fold diluted, and 10⁻⁵, 10⁻⁶, and 10⁻⁷ diluted samples were selected, coated, and anaerobically cultivated at 37° C. for 48 h; colonies with different morphologies were selected and subjected to streak purification, and single colonies were picked, inoculated into an MRS broth medium, and cultivated at 37° C. for 24 h; and all Gram-positive and catalase-negative strains were subcultivated for three generations and then cryopreserved in a skimmed milk at −80° C., such that the Lactobacillus IMAU80584 was finally screened out.

II. Determination of Colony Characteristics, Morphological Characteristics, Physiological and Biochemical Characteristics, and Enzyme Activity for the Lactobacillus IMAU80584

The Lactobacillus IMAU80584 (Lentilactobacillus rapi subsp. dabitei) was inoculated into an MRS broth liquid medium, cultivated at 37° C. for 24 h, and then subcultivated for 3 generations to restore the activity of the strain; a resulting bacterial solution was 10-fold diluted to obtain a 10⁻¹ to 10⁻⁸ dilution gradient; and 200 μL of each of 10⁻⁶, 10⁻⁷, and 10⁻⁸ serially-diluted solutions was pipetted, evenly coated on an MRS agar medium plate, and anaerobically cultivated at 37° C. for 48 h.

The MRS medium included the following components in weight: peptone (10 g), beef extract (10 g), yeast powder (5 g), glucose (20 g), Tween-80 (1 g), dipotassium phosphate (DKP) (2 g), sodium acetate (5 g), sodium citrate (2 g), magnesium sulfate heptahydrate (200 mg), and manganese sulfate pentahydrate (54 mg). The above components were mixed and sterilized at 121° C. for 15 min to obtain the MRS medium.

1) A colony morphology of the Lactobacillus IMAU80584 on a petri dish and a size, color, transparency, colony surface state, and colony edge state of a formed colony were observed and recorded.

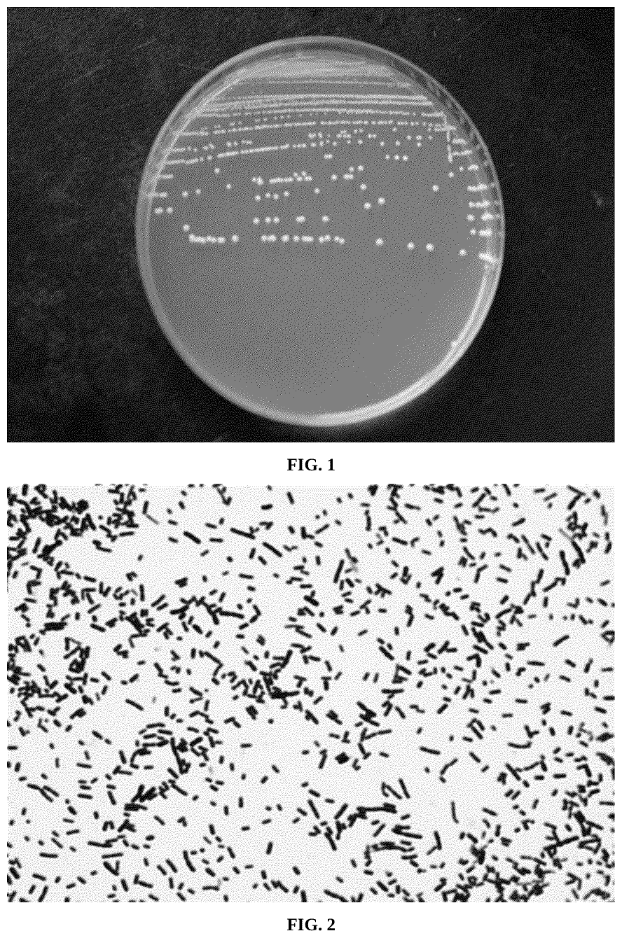

Results showed that a single colony formed by the Lactobacillus IMAU80584 had a diameter of 1 mm to 2 mm, was beige and opaque, and had a smooth and flat surface with a slightly-rough edge (as shown in FIG. 1).

2) The Lactobacillus IMAU80584 was subjected to Gram staining. The cell morphology was observed with a BX53 optical microscope, the cell morphology of the Gram-negative strain was observed with a transmission electron microscope, the generation of air bubbles was observed with a 3% (v/v) H₂O₂ solution to check the catalase activity, and the cell motility was tested in a semi-solid medium (0.3%), where the semi-solid medium was an MRS agar semi-solid medium.

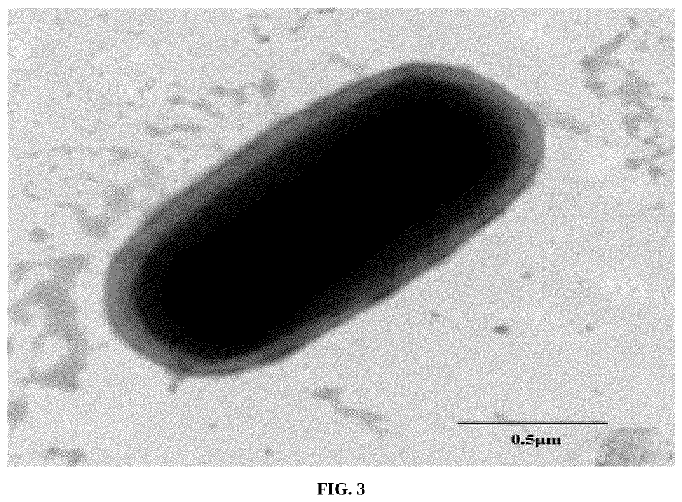

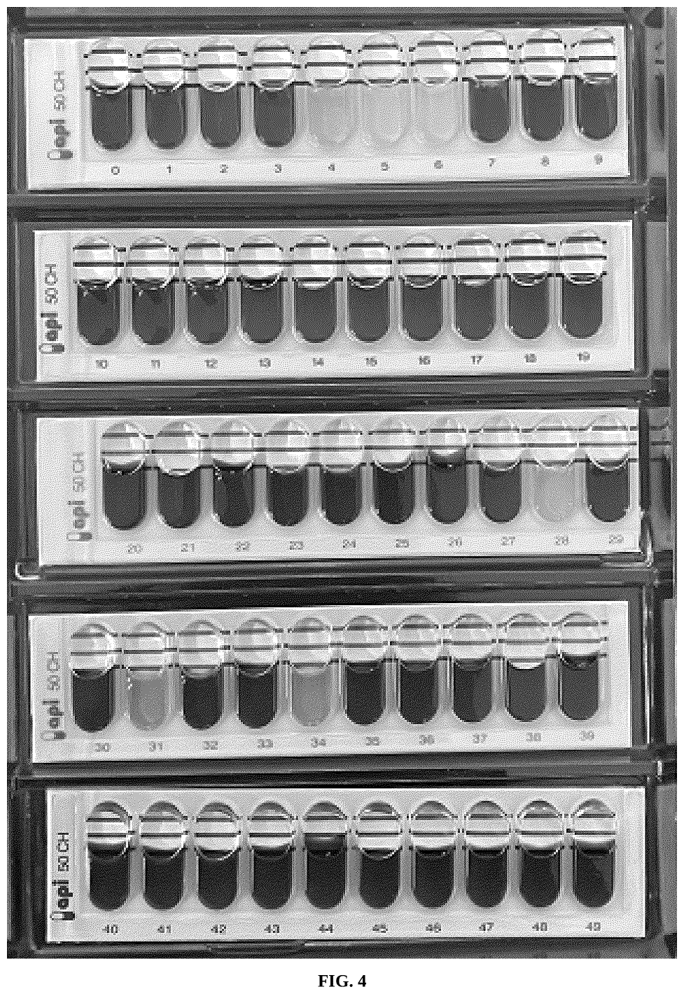

Results showed that the Lactobacillus IMAU80584 was Gram-positive, in a short-rod shape, free of flagella, and immotile (as shown in FIG. 2); a single bacterial cell was rod-shaped, had a length of 1.67 μm to 1.91 μm and a width of 0.67 μm to 0.83 μm, and was in a single-rod arrangement (as shown in FIG. 3); and the Lactobacillus IMAU80584 did not produce bubbles, was catalase-negative, and had no motility.

3) Identification of Physiological and Biochemical Characteristics:

A. A seed culture of the Lactobacillus IMAU80584 was streaked on an MRS agar medium by the three-line method and cultivated at 37° C. for 48 h. According to instructions, a raw material with an appropriate optical density required by the above process was prepared as a cell inoculum in a suspension. The MRS agar medium included peptone: 10.0 g, beef extract: 10.0 g, yeast extract: 5.0 g, diammonium hydrogen citrate ((NH₄)₂HC₆H₅O₇): 2.0 g, glucose (C₆H₁₂O₆·H₂O ): 20.0 g, Tween 80: 1.0 mL, sodium acetate (CH₃COONa₃H₂O): 5.0 g, DKP (K₂HPO₄·3H₂O): 2.0 g, magnesium sulfate (MgSO₄·7H₂O): 0.50 g, manganese sulfate (MnSO₄HZO): 0.25 g, agar: 15.0 g, and distilled water: 1,000 mL, and had a pH of 6.2 to 6.6.

B. The prepared cell inoculum was inoculated into a biochemical identification tube and then aerobically cultivated at 37° C. for 24 h and 48 h, and reaction results were recorded. Positive and negative tests were evaluated according to a color code indicated by an Application Programming Interface (API) system.

TABLE 1
Detection results of metabolism of API50 CHL carbohydrates in the
Lactobacillus IMAU80584 and closely-related Lactobacillus

| | A | | B | | C | | D | |
|---|---|---|---|---|---|---|---|---|
| Property | 24 h | 48 h | 24 h | 48 h | 24 h | 48 h | 24 h | 48 h |
| Mannitol | − | − | − | − | − | − | − | − |
| Erythritol | − | − | − | − | − | − | − | − |
| D-arabinose | − | − | − | − | − | − | − | − |
| L-arabinose | + | + | | + | + | + | + | + |
| D-ribose | + | + | + | + | + | + | − | + |
| D-xylose | + | + | + | + | − | + | + | + |
| L-xylose | − | − | − | − | − | − | − | − |
| D-adonitol 1 | − | − | − | − | − | − | − | − |
| Methyl β-D xylopyranoside | − | − | − | − | − | + | − | − |
| D-galactose | − | − | − | − | − | − | − | − |
| D-glucose | − | − | − | − | − | − | − | + |
| D-fructose | − | − | − | − | − | − | + | + |
| D-mannose | − | − | − | − | − | − | − | − |
| L-sorbose | − | − | − | − | − | − | − | − |
| L-rhamnose | − | − | − | − | − | − | − | − |
| Dulcitol | − | − | − | − | − | − | − | − |
| Inositol | − | − | − | − | − | − | − | − |
| Mannitol | − | − | − | − | − | − | − | − |
| Sorbitol | − | − | − | − | − | − | − | − |
| Methyl-α-D-mannopyranoside | − | − | − | − | − | − | − | − |
| Methyl-α-D-glucopyranoside | − | − | − | − | − | − | − | − |
| N-Acetyl glucosamine (NAG) | − | − | − | − | − | − | − | − |
| Amygdalin | − | − | − | − | − | − | − | − |
| ARBULIN | − | − | − | − | − | − | − | − |
| Esculin-iron citrate | + | + | + | + | + | + | + | + |
| Salicin | − | − | − | − | − | − | − | − |
| D-cellobiose | − | − | − | − | − | − | − | − |
| D-maltose | − | + | − | + | − | − | − | + |
| D-lactose | − | − | − | − | − | − | − | − |
| D-melibiose | − | − | − | − | − | − | − | − |
| D-sucrose | + | + | − | + | − | − | − | + |
| D-trehalose | − | − | − | − | − | − | − | − |
| Inulin | − | − | − | − | − | − | − | − |
| D-melezitose | + | + | − | + | − | − | − | − |
| D-raffinose | − | − | − | − | − | − | − | − |
| Starch | − | − | − | − | − | − | − | − |
| Glycogen | − | − | − | − | − | − | − | − |
| Xylitol | − | − | − | − | − | − | − | − |
| D-gentiobiose | − | − | − | − | − | − | − | − |
| D-Toulon sugar | − | − | − | − | − | − | − | − |
| D-lyxose | − | − | − | − | − | − | − | − |
| D-tagatose | − | − | − | − | − | − | − | − |
| D-fucose | − | − | − | − | − | − | − | − |
| L-fucose | − | − | − | − | − | − | − | − |
| D-arbaitol | − | − | − | − | − | − | − | + |
| L-arbaitol | − | − | − | − | − | − | − | − |
| Potassium gluconate | − | − | − | + | − | − | − | + |
| 2-Keto-potassium gluconate | − | − | − | − | − | − | − | − |
| 5-Keto-potassium gluconate | − | − | − | − | − | − | − | + |

Notes:
1. “−” indicates negative and “+” indicates positive; and
2. A indicates Lactobacillus IMAU80584, B indicates Lentilactobacillus rapi DSM 19907^{T}, C indicates Lentilactobacillus kisonensis DSM 19906^{T}, and D indicates Lentilactobacillus diolivorans DSM 14421^{T}.

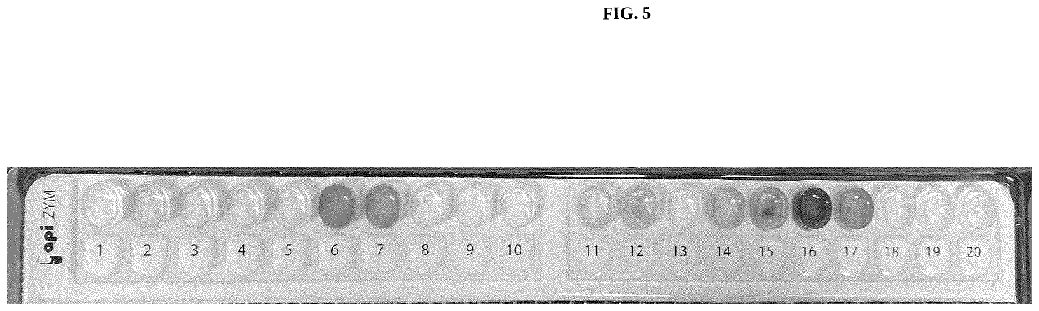

It can be seen from Table 1 and FIG. 4 that the Lactobacillus strain IMAU80584 can use L-arabinose, D-ribose, D-xylose, esculin-iron citrate, D-maltose, D-sucrose, and D-melezitose as a fermentation substrate.

4) Detection of API-ZYM enzyme activity of the Lactobacillus strain IMAU80584

A. A seed culture of the Lactobacillus IMAU80584 was streaked on an MRS agar medium by the three-line method and cultivated at 37° C. for 48 h. According to instructions, a raw material with an appropriate optical density required by the above process was prepared as a cell inoculum in a suspension. The MRS agar medium included peptone: 10.0 g, beef extract: 10.0 g, yeast extract: 5.0 g, diammonium hydrogen citrate ((NH₄)₂HC₆H₅O₇): 2.0 g, glucose (C₆H₁₂O₆·H₂O ): 20.0 g, Tween 80: 1.0 mL, sodium acetate (CH₃COONa₃H₂O): 5.0 g, DKP (K₂HPO₄·3H₂O): 2.0 g, magnesium sulfate (MgSO₄·7H₂O): 0.50 g, manganese sulfate (MnSO₄H₂O): 0.25 g, agar: 15.0 g, and distilled water: 1,000 mL, and had a pH of 6.2 to 6.6.

B. The prepared cell inoculum was inoculated into a biochemical identification tube and then aerobically cultivated at 37° C. for 4 h (a test strip should not be placed under strong light during the cultivation), then a drop of each of ZYMA and ZYMB was added, and 5 min later, a color change of the test strip was observed. Positive and negative tests were evaluated according to a color code indicated by an API system.

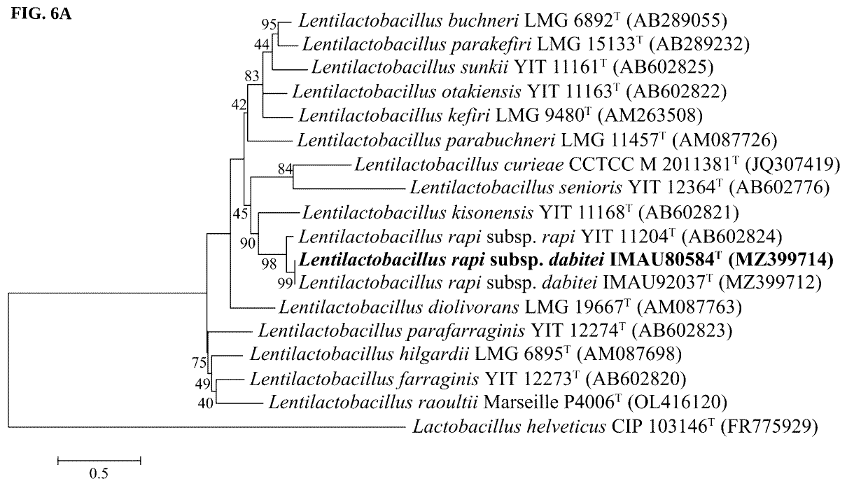

It can be seen from Table 2 and FIG. 5 that the Lactobacillus strain IMAU80584 can express leucine arylamidase, valine arylamidase, naphthol-AS-BI-phosphohydrolase, β-galactosidase, β-glucuronidase, and α-glucosidase.

TABLE 2
Biochemical reaction results of API20 ZYM in the Lactobacillus
IMAU80584 and closely-related Lactobacillus

| | A | B | C | D |
|---|---|---|---|---|
| Control | − | − | − | − |
| Alkaline phosphatase | − | − | − | − |
| Esterase (C4) | − | − | − | − |
| Ester-like esterase (C8) | − | − | − | − |
| Lipoidase (C14) | − | − | − | − |
| Leucine arylamidase | + | + | + | + |
| Valine arylamidase | + | + | + | + |
| Cystine arylamidase | − | − | − | − |
| Trypsin | − | − | − | − |
| Chymotrypsin | − | − | − | − |
| Acid phosphatase | − | − | − | + |
| Naphthol-AS-BI-phosphohydrolase | + | + | + | + |
| α-galactosidase | − | − | + | − |
| β-galactosidase | + | + | − | + |
| β-glucuronidase | + | − | + | + |
| α-glucosidase | + | + | + | + |
| β-glucosidase | − | + | + | + |
| N-acetyl-glucosaminidase | − | − | + | − |
| α-mannosidase | − | − | − | − |
| α-fucosidase | − | − | − | − |

Notes:
1. “−” indicates negative and “+” indicates positive; and 2. A indicates Lactobacillus IMAU80584, B indicates Lentilactobacillus rapi DSM 19907^{T}, C indicates Lentilactobacillus kisonensis DSM 19906^{T}, and D indicates Lentilactobacillus diolivorans DSM 14421^{T}.

III. Identification of 16S rRNA of the Lactobacillus IMAU80584

Single clones of the Lactobacillus IMAU80584 were picked, inoculated into an MRS broth medium, and cultivated in a constant-temperature anaerobic incubator at 37° C. for 24 h until the strain grew to a later logarithmic phase, and then a bacterial paste was collected. The genomic DNA (gDNA) of the strain was extracted using a bacterial gDNA extraction kit according to the manufacturer's instructions, and the universal bacterial 16S rRNA primers 27F: 5′-AGAGTTTGACCTGGCTAG-3′ (SEQ ID NO: 1) and 1495R: 5′-CTACGGCTCCTTGTTCGA-3′ (SEQ ID NO: 2) were used to conduct colony PCR amplification. The DNA quality was detected through gel electrophoresis (0.1% w/v agarose gel), the concentration was determined through spectrophotometry, and a qualified PCR product was sent to Shanghai Sunny Biotechnology Co., Ltd. for 16S rRNA sequencing.

The 16S rRNA sequence of the Lactobacillus IMAU80584 was aligned with 16S rRNA sequences of all determined bacteria in a database on the National Center for Biotechnology Information (NCBI). The 16S rRNA sequence of the strain IMUA80584 had the highest homology with that of Lentilactobacillus rapi DSM 19907T of Lentilactobacillus, and had relatively-low homology with that of other strains. Thus, the strain IMAU80584 was determined to be the genus of Lentilactobacillus.

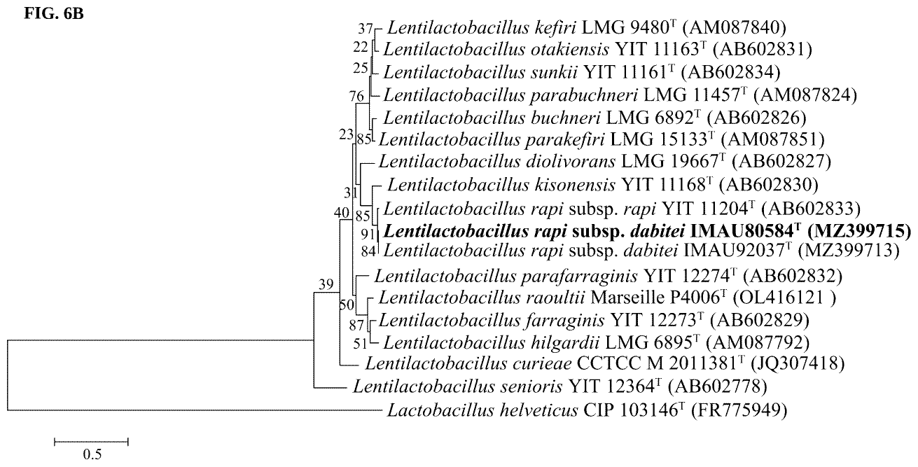

Then, the sequences of two housekeeping genes pheS and rpoA (encoding a subunit of phenylalanyl-tRNA synthase and a subunit of RNA polymerase, respectively) were obtained from the genome sequence, and a phylogenetic tree of the Lactobacillus IMAU80584 and all type strains of sibling species based on the two housekeeping genes (rpoA and pheS) (as shown in FIG. 6A-B) was constructed using the neighbor-joining and maximum likelihood methods and analyzed based on 1,000 replicates using MEGA 7.0 software.

In conclusion, according to the sequence analysis of the 16S rRNA and housekeeping genes and the construction of the phylogenetic tree, the strain IMAU80584 is a suspected new species in the genus of Lentilactobacillus.

IV. Whole-Genome Sequencing (WGS) Identification of the Lactobacillus Strain IMAU80584

The Lactobacillus strain IMAU80584 was inoculated into an MRS broth medium, cultivated at 37° C. for 24 h, and subcultivated for 3 generations to obtain a pure culture of the strain, which was sent to Beijing Novogene Bioinformatics Technology Co. Ltd. for WGS. Illumina Novaseq 6000 was adopted as a genome sequencing platform, and an Illumina PE library was constructed. The genome sequencing data of the obtained strain were subjected to quality assessment and filtered, then high-quality reads were assembled using the software package SOAPdenovo (v2.04), appropriate kmer values were selected and assembled, and the filtered data were subjected to single-base correction. Assembly results were evaluated with genome size, scaffold number, N50 length, N90 length, and GC content as indexes.

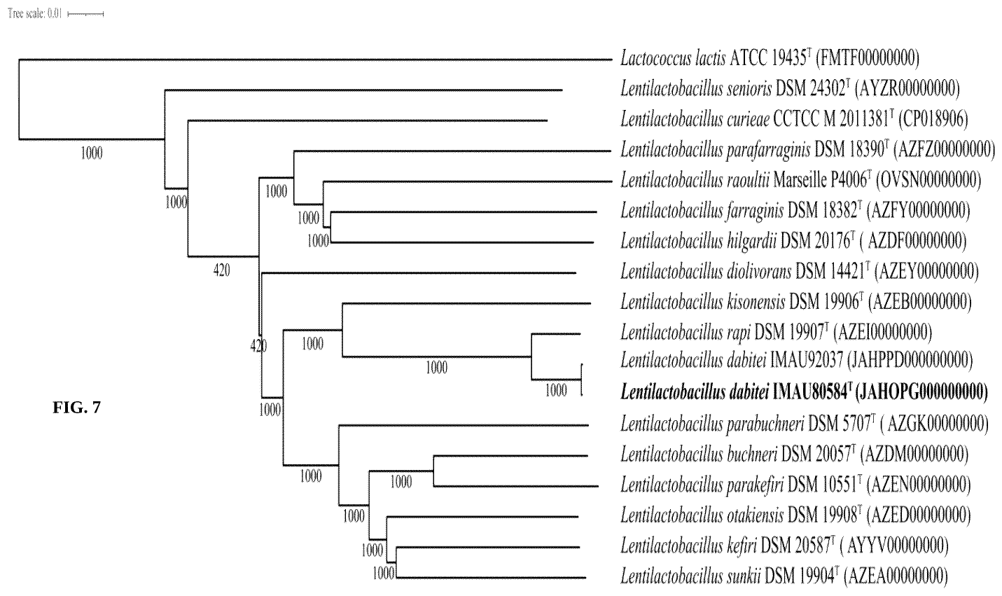

A phylogenetic tree was constructed based on the 88 core genes of all type strains of the Lentilactobacillus genus published by NCBI, and it was found that the Lactobacillus (Lentilactobacillus rapi subsp. dabitei) IMAU80584 and Lentilactobacillus rapi DSM 19907T were clustered together, but independently formed a sub-branch (FIG. 7), indicating that the strain IMAU80584 was a new species in the Lentilactobacillus genus.

The results show that average nucleotide identity (ANI) and genome-to-genome distance calculator (GGDC) values between the Lactobacillus IMAU80584 and Lentilactobacillus rapi DSM 19907T are 93.14 and 52.8, respectively, which are lower than thresholds for defining a species. It can be seen from FIG. 7 that the Lactobacillus IMAU80584 and Lentilactobacillus rapi DSM 19907T are clustered together, but independently form a sub-branch, indicating that the strain IMAU80584 is a new species in the Lentilactobacillus genus.

### Example 2

Detection of growth characteristics of the Lactobacillus IMAU80584

The Lactobacillus IMAU80584 was inoculated into a sterilized MRS broth medium, anaerobically cultivated at 37° C. for 24 h, and then taken out for later use.

I. pH Tolerance Test

A seed culture of the Lactobacillus IMAU80584 was inoculated at an inoculum size of 2% into MRS broth media with pH of 2.5, 3.5, 4.5, 5.5, 6.5, 7.5, 8.5, and 9.5 respectively and anaerobically cultivated at 37° C. for 24 h. The absorbance (OD₆₀₀) was determined at 0 h and 24 h, and the growth of the strain was determined according to an increase in absorbance.

TABLE 3
pH tolerance of the Lactobacillus IMAU80584

| pH | 2.5 | 3.5 | 4.5 | 5.5 | 6.5 | 7.5 | 8.5 | 9.5 |
|---|---|---|---|---|---|---|---|---|
| IMAU80584 | − | W | + | + | + | + | + | W |

Notes:
− indicates no growth;
+ indicates growth;
and w indicates weak growth.
The results in Table 3 show that the Lactobacillus IMAU80584 grows robustly at pH 4.5, 5.5, 6.5, 7.5, and 8.5, grows slowly at pH 3.5 and 9.5, and does not grow at pH 2.5.

II. NaCl Tolerance Test

A seed culture of the Lactobacillus IMAU80584 was inoculated at an inoculum size of 2% into MRS broth media with NaCl concentrations of 2%, 3%, 4%, 5%, 6%, 7%, 8%, 9%, and 10% respectively and anaerobically cultivated at 37° C. for 24 h. The absorbance (OD₆₀₀) was determined at 0 h and 24 h, and the growth of the strain was determined according to an increase in absorbance.

TABLE 4
NaCl tolerance of the Lactobacillus IMAU80584

| NaCl | 2% | 3% | 4% | 5% | 6% | 7% | 8% | 9% | 10% |
|---|---|---|---|---|---|---|---|---|---|
| IMAU80584 | + | + | + | + | W | − | − | − | − |

Notes:
− indicates no growth;
+ indicates growth;
and w indicates weak growth.

The results in Table 4 show that the Lactobacillus IMAU80584 grows weakly in an MRS broth medium with a NaCl concentration of 6%, and basically does not grow in MRS broth media with a NaCl concentration of more than 6%.

III. Temperature Tolerance Test

A seed culture of the Lactobacillus IMAU80584 was inoculated at an inoculum size of 2% into an MRS broth medium and anaerobically cultivated at each of 4° C., 10° C., 20° C., 30° C., 37° C., and 45° C. for 24 h. The absorbance (0D600) was determined at 0 h and 24 h, and the growth of the strain was determined according to a change in absorbance.

TABLE 5
Temperature tolerance of the Lactobacillus IMAU80584

| Temperature (° C.) | 4 | 10 | 20 | 30 | 37 | 45 |
|---|---|---|---|---|---|---|
| IMAU80584 | − | + | + | + | + | + |

Notes:
− indicates no growth;
+ indicates growth;
and w indicates weak growth.

The results in Table 5 show that the Lactobacillus IMAU80584 does not grow at 4° C., but grows at 10° C. to 45° C.

### Example 3

Determination of fermentation ability of the Lactobacillus IMAU80584

1. Soybean Milk Fermentation

1) Preparation Method of a Soybean Milk Fermentation Solution

A soybean powder with a protein content of 42% was used to prepare a soybean milk fermentation solution with a protein content of 5%, 6% of sucrose was added, a resulting mixture was evenly dispensed into 100 mL conical flasks with 40 mL in each conical flask, and the conical flasks were sealed, sterilized at 121° C. for 15 min, and rapidly cooled to 40° C. for later use.

2) Method for Fermenting Soybean Milk with the Lactobacillus IMAU80584

A seed culture of the Lactobacillus IMAU80584 was cultivated overnight in an MRS broth medium and then inoculated at an inoculum size 8% of a soybean milk volume into the prepared soybean milk fermentation solution, and then fermentation was conducted at 37° C. for 24 h.

3) Evaluation of Acid-Producing Ability of the Lactobacillus IMAU80584 when Fermenting Soybean Milk

After the inoculation, a pH of the soybean milk was determined at different fermentation time points of 0 h, 3 h, 6 h, 9 h, 12 h, 15 h, 18 h, 21 h, and 24 h.

The results show that the fermented soybean milk has a pH of 6.4 at the initial fermentation time point, and the pH of the fermented soybean milk gradually decreases with the extension of fermentation time; and the pH of the fermented soybean milk reaches a minimum of 4.7 at the fermentation time point of 15 h, and then tends to be stable.

2. Cow Milk Fermentation

1) Preparation Method of a Cow Milk Fermentation Solution

A skimmed milk powder was used to prepare a 89% cow milk fermentation solution, 6% of sucrose was added, a resulting mixture was evenly dispensed into 100 mL conical flasks with 40 mL in each conical flask, and the conical flasks were sealed, sterilized at 121° C. for 15 min, and rapidly cooled to 40° C. for later use.

2) Method for Fermenting Cow Milk with the Lactobacillus IMAU80584

A seed culture of the Lactobacillus IMAU80584 was cultivated overnight in an MRS broth medium and then inoculated at an inoculum size 8% of a cow milk volume into the prepared cow milk fermentation solution, and then fermentation was conducted at 37° C. for 24 h.

3) Evaluation of Acid-Producing Ability of the Lactobacillus IMAU80584 when Fermenting Cow Milk

After the inoculation, a pH of the cow milk was determined at different fermentation time points of 0 h, 3 h, 6 h, 9 h, 12 h, 15 h, 18 h, 21 h, and 24 h.

The results show that the fermented cow milk has a pH of 6.1 at the initial fermentation time point, and the pH of the fermented cow milk gradually decreases with the extension of fermentation time; and the pH of the fermented cow milk reaches a minimum of 4.6 at the fermentation time point of 15 h, and then tends to be stable.

### Example 4

Detection of Antibacterial Ability of the Lactobacillus IMAU80584

E. coli was selected as a pathogenic indicator bacterium.

An experimental process was conducted in accordance with the national standard GB/T 39101-2020, and specific operations were as follows:

1) A seed culture of the Lactobacillus IMAU80584 was taken, inoculated at an inoculum size of 2% into a liquid medium, and cultivated at 37° C. for 24 h, 500 μL of a resulting bacterial solution was taken and centrifuged, a resulting supernatant was taken and filtered through a 0.22 μm microporous membrane, and the remaining bacterial cell pellet was resuspended with the same volume of sterile normal saline (NS) for later use.

2) The antibacterial activity of the Lactobacillus IMAU80584 was determined by the Oxford cup method.

The indicator bacterium was inoculated into a test tube with 5.0 mL of an NB medium and subcultivated for three generations at 37° C. to obtain a bacterial solution; the prepared nutrient agar (NA) medium was heated for melting and cooled to 45° C. to 50° C.; the prepared suspension of the indicator bacterium was diluted to a gradient of 10⁻⁴ and added at an amount of 7% to the NA medium, a resulting mixture was thoroughly mixed and poured into a sterilized Petri dish, and the Petri dish was gently shaken for even spreading and then allowed to stand for solidification; and 3 Oxford cups were placed at an equal distance on the test dish with the indicator bacterium, and 100 μL of each of a mixed bacterial solution and a bacterium-free supernatant of the Lactobacillus IMAU80584 was measured and slowly added to the Oxford cup. 3 replicates were set for each treatment and 3 parallel experiments were conducted for each dish.

4. The dish with the mixed bacterial solution was placed in a 37° C. constant-temperature incubator, and cultivated upright until clear inhibition zones appeared. A diameter of each of the inhibition zones was measured by an inhibition zone measuring instrument, where each inhibition zone was measured three times in different directions, and an average value was taken.

5. The dish with the bacterium-free supernatant was transferred to a 4° C. refrigerator to allow pre-diffusion for 8 h, then taken out and placed in a 37° C. constant-temperature incubator, and cultivated upright until clear inhibition zones appeared. A diameter of each of the inhibition zones was measured by an inhibition zone measuring instrument, where each inhibition zone was measured three times in different directions, and an average value was taken. Results were shown in Table 6.

TABLE 6
Antibacterial results of the Lactobacillus IMAU80584

| Sample | Inhibition zone size |
|---|---|
| Mixed bacterial solution | 11-12 mm |
| Supernatant | 14-15 mm |

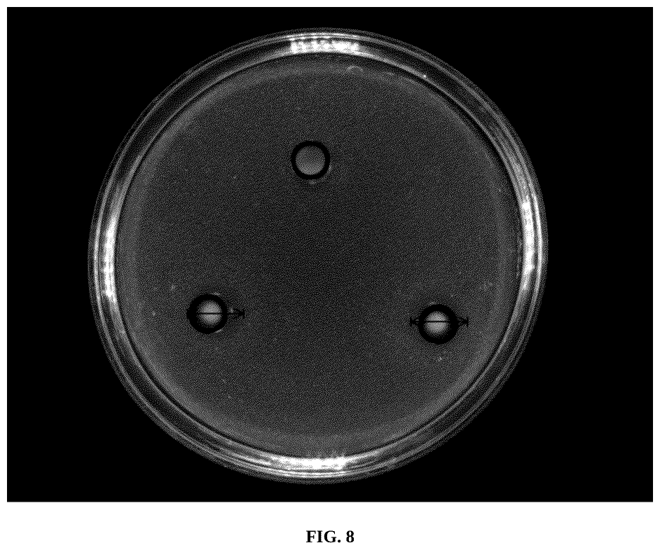

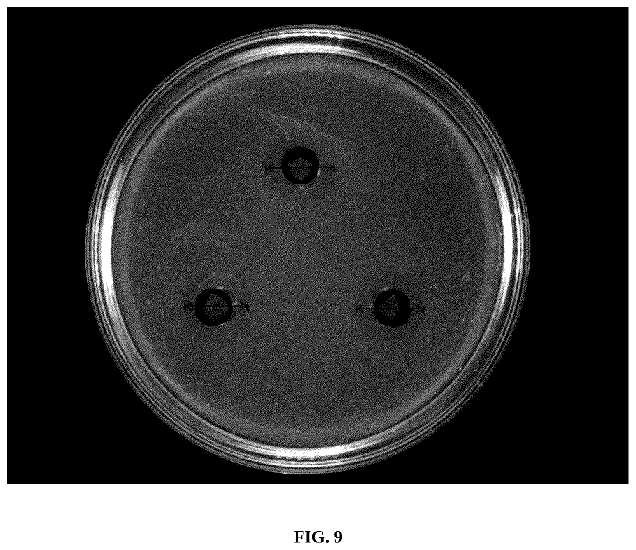

It can be seen from the antibacterial results in FIG. 8, FIG. 9, and Table 6 that the Lactobacillus strain IMAU80584 of the present disclosure has a relatively-significant antibacterial effect on E. coli. Therefore, the Lactobacillus IMAU80584 of the present disclosure has excellent development potential in the preparation of a product for preventing and treating pathogenic bacteria.

The above are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A fermentation microbial agent comprising a Lactobacillus strain IMAU80584, where in the Lactobacillus strain IMAU80584 is deposited in the Guangdong Microbial Culture Collection Center (GDMCC), with an accession number of GDMCC 1.2566

2. The fermentation microbial agent according to claim 1, wherein an effective viable concentration of the Lactobacillus strain IMAU80584 is 1×10⁶ CFU/mL to 1×10⁸ CFU/mL.

3. A method for preparing a fermented milk, comprising the fermentation microbial agent according to claim 1.

4. The method according to claim 3, wherein an effective viable concentration of the Lactobacillus strain IMAU80584 is 1×10⁶ CFU/mL to 1×10⁸ CFU/mL.

5. The method according to claim 3, wherein the fermented milk comprises a fermented soybean milk or cow milk.

6. The method according to claim 4, wherein the fermented milk comprises a fermented soybean milk or cow milk.

7. A product for preventing and treating pathogenic bacteria, comprising a Lactobacillus strain IMAU80584.

8. The product according to claim 7, wherein the pathogenic bacteria comprise Escherichia coli (E. coli).
